(19) United States
(12) Patent Application Publication
(10) Pub. No.: US 2004/0047831 A1
(43) Pub. Date: Mar. 11, 2004

(54) Use of a cell extract of at least a plant of the family pontederiaceae as anti-pollution agent

(76) Inventors: Richard Martin, Rochecorbon (FR); Pascal Hilaire, Vouvray (FR); Beatrice Belcour-Castro, La Riche (FR); Philippe Catroux, Nogent-sur-Marne (FR); Jose Cotovio, Danmartin-en-Goele (FR)

Correspondence Address: D Douglas Price, Steptoe & Johnson, 1330 Connecticut Avenue NW, Washington, DC, 20036, US

(21) Appl. No.: 10/276,543

(22) PCT Filed: May 14, 2001
(86) PCT No.: PCT/FR01/01450

(30) Foreign Application Priority Data
May 18, 2000 (FR) 00/06385

(51) Int. Cl. A61K 7/06; A61K 35/78

(52) U.S. Cl. 424/74; 424/725

(57) ABSTRACT

The invention concerns the use of a cell extract of at least a plant of the family Pontederiaceae as anti-pollution or depollution agent, conxpositions containing at least one of said extracts, and a cosmetic treatment method using said cell extracts or said compositions.

The present invention relates to the use of extracts of cells of at least one plant from the Pontederiacea family as antipollution and depollution agents, and also to compositions containing at least one of these extracts. The invention also relates to a cosmetic treatment process using the cell extracts or the compositions.

Metal ions are required by the body in the form of trace amounts as essential nutrients. For example, several functions involving polypeptides, such as enzymatic, structural and immunological functions, require metallic cofactors.

However, other metal ions, in particular heavy metal ions when they are at nonphysiological concentrations, may impair these functions. Thus, overexposure to metals of the environment can lead to toxic effects.

Ecological studies conducted in industrialized countries show that the amounts of metals present in the atmosphere are on the increase. This leads to an increase in the levels of heavy metals in body tissues following the ingestion of contaminated food and exposure to atmospheric metals.

The effects of accumulation of heavy metals may be extremely hazardous and their toxicity is partially due to the impairment of the tertiary and quaternary structures of proteins, which results in a reduction in their catalytic activity. The impaired proteins may become antigenic and bring about an immune response. They are then recognized by the body as foreign polypeptides and may give rise to autoimmune responses.

Another mechanism responsible for the toxic effects of metals is the competitive substitution of natural physiological cofactors with heavy metals at nonphysiological concentrations. Thus, controlling the pollutant heavy metals in the atmosphere is essential for preventing diseases in relation with exposure to metals.

Due to the increasing contamination of the environment with heavy metals and their ubiquitous presence in the ecosystem, the skin, the hair and the accessible mucous membranes represent the largest area of contact and thus promote the accumulation of metals and their subsequent absorption into the body.

Certain metals and metal compounds present in industrial manufactured products, chemicals, jewelry, clothing, medicinal products, colorants and cleaning products are involved in primary irritation reactions, allergic reactions and carcinogenicity reactions in skin tissue.

The metals that are the main offenders -in the environment are copper, cobalt, zinc, manganese, mercury, nickel, lead and cadmium.

Skin rashes caused by metal-induced dermatitis are a problem encountered in people exposed to large amounts of certain metal ions. Exposure to nickel in the environment is largely due to the frequent use of this metal in jewelry articles, watch straps and clothing buttons. Sensitization to nickel with the development of dermatitis is an industrial hazard in certain occupations. Moreover, the deposition of minerals on the hair is an inevitable phenomenon.

The hair is a strong absorber of metals. The binding is so strong that once these bound metals have been captured by the anionic sites of the fiber, they are difficult to elute. The degree of binding of metals to the hair generally depends on several factors such as the size of the fiber, its porosity and the exposure time. Metals such as copper, lead and iron may interfere with chemical treatments such as the dyeing and permanent waving of the hair.

Certain cosmetic products contain metals such as magnesium, copper or iron. The absorption of these metals by keratin fibers may interfere with chemical treatments such as dyeing, bleaching or permanent-waving effects. These interactions may lead to problems in dyeing or precipitations, as described in American patent U.S. Pat. No. 5,635,167.

It has been demonstrated that certain heavy metals penetrate into the skin and are accumulated (A. B. G. Landsdown. Critical Reviews in Toxicology, 1995, 25:397-462). At high concentrations, they can induce:

oxidation mechanisms on membrane lipids,

direct cytotoxicity, liable to result in cellular necrosis,

alkylation of the cellular nucleophiles, which may be the cause of sensitization phenomena or carcinogenesis.

(S. J. Stochs and D. Bagchi. Free Radical Biology and Medicine. 1995, 18:321-336; M. E. Figueiredo Pereira et al., The Journal of Biological Chemistry. 1998, 21:12703-12709; N. L. Acan et al., 1995, Biochemical and Molecular Medicine. 54:33-37.)

Cosmetic and therapeutic treatments have already been envisioned, by protecting tissues with:

compounds with sulfur-containing groups which behave like heavy metal sequestering agents, for instance the metallothioneines in patent EP 0 557 042 A1, and

amino acid compounds with sulfur-containing groups in patent application EP 0 914 815 A1.

Moreover, patent application GB 2 333 705 A mentions the use of ethylenediaminedisuccinic acid in compositions for treating heavy-metal-induced skin irritations.

The problem posed is thus that of protecting the skin against metal particles floating in the air and the deleterious effects thereof encountered in urban pollution, by selecting active principles that are effective in controlling these effects and/or preventing the penetration of metals into the skin or keratin materials, and by introducing these active principles into cosmetic formulations.

It has now been found, entirely surprisingly, that the use of an extract of cells of at least one plant from the Pontederiacea family allows keratin materials, the skin and integuments to be protected against the effects of a certain category of atmospheric pollutants, for instance heavy metals.

One subject of the present invention is the use of an extract of cells of at least one plant from the Pontederiacea family as an antipollution or depollution cosmetic agent.

The expression “antipollution cosmetic agent” means an agent that protects the skin and keratin materials so as to prevent, attenuate and/or eliminate the deleterious effects of metals.

A subject of the present invention is also cosmetic compositions containing such cell extracts, and also an antipollution or depollution cosmetic treatment process using these compositions or Pontederiacea cell extracts.

The Applicant has discovered that this model of plants accumulates a broad spectrum of heavy metals, thus giving the best possible coverage of all the heavy metals considered hazardous to the skin.

A subject of the invention is the use of an extract of cells of at least one plant from the Pontederiacea family as an antipollution or depollution cosmetic agent.

The Pontederiacea family includes several genera, among which is Eichhornia crassipes, also known as water hyacinth. This genus contains about seven species, including E. azurea and E. crassipes. These are aquatic or amphibious floating weeds with a procumbent rhizome and floating or submerged sheating leaves.

A subject of the present invention is more particularly the use of cell extracts obtained from Eichhornia crassipes.

The cell extracts are preferably root or leaf cell extracts.

Any extraction method known to those skilled in the art may be used according to the invention. Mention may be made in particular of alcoholic extracts, especially ethanolic and/or methanolic extracts, and aqueous-alcoholic extracts.

An extract prepared by the method described in French patent application No. 95/02379 may also be used. Thus, in a first step, the plant material is ground in a cold aqueous solution, in a second step, the particles in suspension are removed from the aqueous solution obtained from the first step, and in a third step, the aqueous solution obtained from the second step is sterilized. This aqueous solution corresponds to the extract.

Moreover, the first step may advantageously be replaced with an operation of simple freezing of the plant tissues (for example at −20° C.), followed by an aqueous extraction that repeats the second and third steps described above.

Preferably, according to the invention, an ethanolic extract is used.

Irrespective of the method for preparing the extract, said extract may then be freeze-dried for possible storage. An example of an extract preparation that may be used according to the invention is given later in the examples.

One subject of the present invention is the use of at least one cell extract as an antipollution or depollution cosmetic agent in cosmetic or dermatological compositions.

These cell extracts may be used as antipollution or depollution agents, preferably as antipollution or depollution cosmetic agents, to protect keratin materials, integuments and the skin against the harmful effects of heavy metals.

The integuments represent the nails, the eyelashes, mucous membranes, the eyebrows and body hairs in general.

These cell extracts may be used as antipollution agents, preferably as antipollution or depollution cosmetic agents, to improve cell respiration and/or to reduce desquamation and/or to prevent keratin materials, the skin or integuments from looking dull or being soiled.

Another subject of the present invention is the preparation of a cosmetic composition with at least one cell extract defined previously.

Said composition may contain from 0.01% to 10% and preferably 0.1% to 5% by weight of cell extracts relative to the total weight of the composition.

This composition may also contain at least one other antipollution compound.

Said antipollution compound may be chosen especially from anthocyans and/or derivatives thereof, compounds containing a thioether, sulfoxide or sulfone function, ergothioneine and/or its derivatives, metal-chelating agents such as, for example, N,N′-dibenzylethylene-diamine-N,N′-diacetic acid derivatives, and antioxidants.

Among the antioxidants that will be chosen more particularly are polyphenols and, inter alia, ellagic acid.

The cosmetic compositions used according to the invention also comprise a cosmetically acceptable medium more particularly consisting of water and/or optionally of a cosmetically acceptable organic solvent.

They may be chosen from the group consisting of hydrophilic organic solvents, amphiphilic solvents and lipophilic organic solvents, or mixtures thereof.

Among the hydrophilic organic solvents that may be mentioned, for example, are linear or branched lower monoalcohols containing from 1 to 8 carbon atoms, for instance ethanol, propanol, butanol, isopropanol and isobutanol, polyethylene glycols containing from 6 to 80 ethylene oxides, polyols such as propylene glycol, isoprene glycol, butylene glycol, glycerol, sorbitol, monoalkyl or dialkyl isosorbides, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether.

Amphiphilic organic solvents that may be mentioned include polyols such as propylene glycol (PPG) derivatives, such as esters of polypropylene glycol and of fatty acids, derivatives of PPG and of fatty alcohols, for instance PPG-23 oleyl ether, and PPG-36 oleate.

Lipophilic organic solvents that may be mentioned, for example, include fatty esters such as diisopropyl adipate, dioctyl adipate and alkyl benzoates.

The organic solvents are preferably chosen from mono-functional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

The organic solvents may represent from 5% to 98% of the total weight of the composition.

In order for the compositions used in the invention to be more pleasant to use, softer to apply, more nourishing and more emollient, it is possible to add a fatty phase to the medium of these compositions.

The fatty phase preferably represents from 0 to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grapeseed oil, rapeseed oil or coconut oil,

synthetic oils such as purcellin oil and isoparaffins,

fluoro oils and perfluoro oils,

fatty acid esters such as purcellin oil.

Said fatty phase may also comprise as fatty substances one or more fatty alcohols, fatty acids or waxes (paraffin wax, polyethylene wax, carnauba wax or beeswax).

In a known manner, the compositions used in the invention may also contain adjuvants that are common in cosmetics, such as standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and anti-dandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions used according to the invention may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, these spherules possibly being polymer nanoparticles such as nanospheres and nanocapsules, or better still lipid vesicles of ionic and/or nonionic type.

The compositions used in the present invention may be more or less fluid and may have the appearance of a white or colored cream, an ointment, a milk, a lotion, a serum, a paste, a mousse or a solid.

They may optionally be applied to the skin in aerosol form.

They may also be applied in solid form, and for example in the form of a stick.

They may be used as care products and/or as makeup products.

The compositions of the invention may have a pH of between 3 and 8 and preferably between 5 and 7.

A subject of the present invention is also the use of a cell extract in or for the preparation of an antipolluting or depolluting composition.

A subject of the invention is the use of such a cosmetic composition for protecting keratin materials, integuments and the skin against the harmful effects of heavy metals, and also for improving cell respiration and/or for reducing desquamation and/or for preventing keratin materials, integuments and the skin from looking dull or being soiled.

Another subject of the invention consists of a cosmetic treatment process for protecting the body against the effects of pollution, which consists in applying to keratin material, integuments or the skin a cosmetically effective amount of at least one cell extract as defined above.

Another cosmetic treatment process according to the invention, for protecting the body against the effects of pollution, consists in applying to keratin material, integuments or the skin a cosmetic composition according to the invention as defined above.

These cosmetic treatment processes may be followed by a rinsing operation after applying cell extracts or cosmetic compositions according to the invention as defined above to keratin material, integuments or the skin.

The examples that follow are intended to illustrate the invention without, however, being limiting in nature.

Preparation of the Plant Extracts

Protocol 1: 185 g of fresh roots are introduced into 800 ml of distilled water or MilliQ-grade water. Coarse grinding is carried out in a domestic food processor for five minutes. This step is completed by fine grinding: Turrax machine for 10 minutes. The decantable portion is separated out by centrifugation at 8000×G/20 minutes or frontal cascade filtration. The final filtration is performed on a Whatmann GFD filter. 621 g of filtrate are thus recovered and then freeze-dried to give 1.26 g of lyophilizate.

Protocol 2: Leaves+Roots: 52.8 g, chopping with scissors, grinding with liquid nitrogen in a mortar, taking up in 200 ml of MilliQ H₂O, magnetic stirring at 900 revolutions/minute for 10 minutes, 180 ml recovered and then filtered through a Whatmann GFF filter, gave 417 mg of lyophilizate.

Protocol 3: 12 water hyacinth plants were washed with water and then crudely drained. After chopping in a knife mill (chopping processor), 700 g of ground material were obtained. Addition of 700 ml of H₂O and then 300 ml of MilliQ H₂O. Further chopping in the chopping processor for five minutes, centrifugation for 20 minutes at 8000×G, Whatmann GFD and then GFF filtration and freeze-drying: 5.43 g of lyophilizate are thus obtained.

Demonstration of the Protective Effect, on Keratinocytes Cultured in vitro, of a Hyacinth Extract with Respect to a Representative Heavy Metal: Cadmium

Principle:

Heavy metals such as cadmium, nickel, lead, mercury, etc. can lead to cytotoxicity on the cells of various organs including the skin. The cytotoxic effect of cadmium on human keratinocytes in culture was evaluated by means of a technique using a fluorescent probe to measure the level of intracellular glutathione (J. C. Fernandez Checa et al. 1990, R. Kannan et al. 1993).

Inoculation of the Cells:

The study was performed on a monolayer culture of human keratinocytes obtained from plastic surgeries. The cells are inoculated on D-3 into 96-well culture dishes at a rate of 25 000 cells/cm² in 100 μl of culture medium (calf serum-free defined medium, NR2, Biofluids). The incubations are performed in an incubator at 37° C. under a humid atmosphere enriched with 5% CO₂.

Treatment with the Pollutant in the Presence of the Protecting Agent:

The cells are treated for 24 hours with increasing concentrations (0, 10, 25, 50, 75, 100, 150 and 200 μM) of cadmium chloride (CdCl₂) alone, so as to determine its cytotoxicity. In parallel, a treatment is performed under the same conditions, but in the presence of hyacinth extract (0.125%). The incubations are performed in an incubator at 37° C., [lacuna]% CO₂ under a humid atmosphere.

Measurement of the Cell Viability:

At the end of the contact period, labeling of the intracellular glutathione is performed using a specific fluorescent probe: monochlorobimane (MCB).

Briefly, after treatment, rinse the cells with buffer (PBS) so as to remove the treatment solutions.

Add 100 μl per well of a 1 mM MCB solution over one hour at room temperature and protected from light.

Eliminate the MCB, and add 200 μl/well of PBS buffer.

Read on the spectrofluorimeter (excitation: 380 nm; emission: 485 nm).

The fluorescence obtained is proportional to the amount of glutathione conjugated to the MCB.

Calculate the concentration of CdCl₂ that results in a 50% drop in the level of glutathione (=IC.50)

Results

Number of independent experiments: 3

Number of repetitions per experiment: 4

The results, given as the concentration resulting in a 50% drop in cell viability, are collated in the following table:

Cytotoxicity of cadmium chloride toward human keratinocytes in culture, in the presence or absence of hyacinth extract

IC.50 of cadmium chloride

| without protecting agent | with protecting agent |
|---|---|
| Mean ± SEM | Mean ± SEM |
| 43.7 ± 1.5 μM | 67.7 ± 0.6 μM |

Conclusion

Cadmium chloride alone shows considerable toxicity, with an IC.50 of 43.7 μM. In the presence of hyacinth extract (0.125%), the cytotoxicity of cadmium chloride decreases significantly (which corresponds to an increase in the IC.50): 67 μM.

Starting with an in vitro biological model using human keratinocytes, this experiment shows that a representative agent of a category of atmospheric pollutants (heavy metals) such as cadmium leads under these experimental conditions to strong toxicity, and that the hyacinth extract significantly protects the cells against the toxicity of this pollutant.

### Formulation Examples

### EXAMPLE 1

According to the usual preparation techniques, the constituents below are mixed together to prepare an emulsion.

COMPOSITION FOT TOPICAL APPLICATION

| | | |
|---|---|---|
| cell extract of Eichhornia crassipes | 3 | g |
| polyethylene glycol oxyethylenated | 3 | g |
| with 50 mol of ethylene oxide | | |
| monodiglyceryl stearate | 3 | g |
| liquid petroleum jelly | 24 | g |
| cetyl alcohol | 5 | g |
| water | qs 100 | g |

### EXAMPLE 2

In the same manner, an emulsion is prepared according to a standard technique, using the following compounds:

| | | |
|---|---|---|
| cell extract of Eichhornia crassipes | 4 | g |
| sorbitol | 10 | g |
| glyceryl isostearate | 4 | g |
| liquid paraffin | 24 | g |
| vitamin E | 1 | g |
| glycerol | 3 | g |
| water | qs 100 | g |

### EXAMPLE 3

Starting with the constituents below, the following composition is formulated:

| | | |
|---|---|---|
| cell extract of Eichhornia crassipes | 1 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| ellagic acid | 0.01 | g |
| stearic acid | 4 | g |
| polyethylene glycol stearate | 3 | g |
| vitamin E | 1 | g |
| glycerol | 3 | g |
| water | qs 100 | g |

### EXAMPLE 4

Tablets

| | | |
|---|---|---|
| cell extract of Eichhornia crassipes | 0.1 | g |
| starch | 0.114 | g |
| dicalcium phosphate | 0.02 | g |
| lactose | 0.06 | g |
| magnesium stearate | 0.005 | g |

After tableting, a 0.2 g tablet is obtained.

### EXAMPLE 5

Drinkable Suspension

| cell extract of Eichhornia crassipes | 0.1 | g |
|---|---|---|
| glycerol | 0.5 | g |
| 70% sorbitol | 0.5 | g |
| L-(+)-ergothioneine in which R = H, | 0.01 | g |
| R′ = ethyl, R″ = H and X = NH2 | | |
| methyl para-hydroxybenzoate | 0.04 | g |
| flavoring | qs | |
| purified water | qs 5 | g |

### EXAMPLE 6

Injectable Ampule

| | | |
|---|---|---|
| cell extract of Eichhornia crassipes | 0.1 | g |
| water for injectable preparation | qs 3 | g |

1. The use of at least one extract of cells of at least one plant from the Pontederiacea family as an antipollution or depollution cosmetic agent.

2. The use as claimed in claim 1, characterized in that the cell extract is obtained from Eichhornia crassipes cells.

3. The use as claimed in claim 1 or 2, characterized in that the cells used are obtained from root or leaf.

4. The use of at least one cell extract as claimed in claims 1 to 3, as an antipollution or depollution cosmetic agent in cosmetic or dermatological compositions.

5. The use of at least one cell extract as claimed in claims 1 to 3, as an antipollution or depollution cosmetic agent for protecting keratin materials, integuments and the skin against the harmful effects of heavy metals.

6. The use of at least one cell extract as claimed in claims 1 to 3, as an antipollution or depollution cosmetic agent for improving cell respiration and/or for reducing desquamation and/or for preventing keratin materials, integuments and the skin from looking dull or being soiled.

7. A cosmetic composition, characterized in that it contains, in a cosmetically acceptable medium, at least one extract of cells of at least one plant from the Pontederiacea family.

8. The composition as claimed in claim 7, characterized in that the cells are obtained from Eichhornia crassipes.

9. The composition as claimed in claim 7 or 8, characterized in that the extract is a root or leaf cell extract.

10. The composition as claimed in any one of claims 7 to 9, characterized in that said composition contains from 0.01% to 10% and preferably 0.1% to 5% by weight of cell extract relative to the total weight of the composition.

11. The composition as claimed in any one of claims 7 to 10, characterized in that said composition also contains at least one other antipollution compound.

12. The composition as claimed in claim 11, characterized in that said antipollution compound is chosen from anthocyans and/or derivatives thereof, compounds containing a thioether, sulfoxide or sulfone function, ergothioneine and/or its derivatives, metal-chelating agents such as, for example, N,N′-dibenzylethylenediamine-N,N′-diacetic acid derivatives, and antioxidants.

13. The composition as claimed in claim 12, characterized in that the antioxidants chosen are preferably polyphenols.

14. The composition as claimed in claim 13, characterized in that the polyphenol chosen is preferably ellagic acid.

15. The composition as claimed in any one of claims 7 to 14, characterized in that the cosmetically acceptable medium consists of water and/or of at least one organic solvent.

16. The composition as claimed in claim 15, characterized in that the organic solvent is chosen from the group consisting of hydrophilic organic solvents, lipophilic organic solvents and amphiphilic solvents, or mixtures thereof.

17. The composition as claimed in either of claims 15 and 16, characterized in that the organic solvents are preferably chosen from monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

18. The composition as claimed in claim 15 or 17, characterized in that the organic solvent(s) represent(s) from 5% to 98% relative to the total weight of the composition.

19. The composition as claimed in any one of claims 7 to 18, characterized in that it also comprises at least one fatty phase.

20. The composition as claimed in claim 19, characterized in that the fatty phase represents from 0 to 50% relative to the total weight of the composition.

21. The composition as claimed in any one of claims 7 to 20, characterized in that it also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

22. The composition as claimed in any one of claims 7 to 21, characterized in that it is in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

23. The composition as claimed in any one of claims 7 to 22, characterized in that it has the appearance of a white or colored cream, an ointment, a milk, a lotion, a serum, a paste, a mousse or a solid.

24. The composition as claimed in any one of claims 7 to 23, characterized in that the composition has a pH of between 3 and 8 and preferably between 5 and 7.

25. The use of a cell extract as defined in any one of claims 1 to 3, in or for the preparation of an antipolluting or depolluting composition.

26. The use of a cosmetic composition as defined in any one of claims 7 to 24, for protecting keratin materials, integuments and the skin against the harmful effects of heavy metals.

27. The use of a cosmetic composition as defined in any one of claims 7 to 24, to improve cell respiration and/or to reduce desquamation and/or to prevent keratin materials, integuments and the skin from looking dull or being soiled.

28. A cosmetic treatment process for protecting the body against the effects of pollution, characterized in that it consists in applying to keratin materials, integuments or the skin a cosmetically effective amount of at least one cell extract as defined in any one of claims 1 to 3.

29. A cosmetic treatment process for protecting the body against the effects of pollution, characterized in that it consists in applying to keratin materials, integuments or the skin a composition as defined in any one of claims 7 to 24.
